(19) United States
(12) Patent Application Publication
(10) Pub. No.: US 2022/0273360 A1
(43) Pub. Date: Sep. 1, 2022

(54) ELECTROSURGICAL SYSTEMS

(71) Applicant: Covidien LP, Mansfield, MA (US)

(72) Inventors: Tingting Ma, Shanghai (CN); Jianguo Mao, Shanghai (CN); Yinan Liu, Shanghai (CN); Jun Zhu, Shanghai (CN); Dan Yuan, Shanghai (CN)

(21) Appl. No.: 17/632,986

(22) PCT Filed: Aug. 7, 2019
(86) PCT No.: PCT/CN2019/099590

§ 371 Date: Feb. 4, 2022

(51) Int. Cl. A61B 18/14

(52) U.S. Cl. CPC A61B 18/1482; A61B 2218/006; A61B 2218/008; A61B 2018/0091

(57) ABSTRACT

An electrosurgical system (10) includes an electrosurgical instrument (16) coupled to a gas control unit (14). The gas control unit (14) supplies a preset flow rate of gas to the electrosurgical instrument (16) for displacing surgical smoke and/or blood from a surgical site. A vent tube may be coupled to the gas control unit (14) for regulating a pressure within the surgical site.

### BACKGROUND

The disclosure relates to electrosurgical systems and, more particularly, to electrosurgical systems including an electrosurgical instrument and a means for improving visualization of a surgical site.

Laparoscopic surgery sometimes involves the insertion of a probe through an abdominal wall. The probe may deliver electrosurgical energy into an abdominal cavity to treat selected tissue therein. During the treatment of tissue using the probe, surgical smoke and/or blood may inhibit a clinician's visualization of the surgical site. This lack of visibility can lengthen the procedure, adding to costly operating room time and subjecting the patient to increased time under anesthesia.

Devices employing a system for suctioning the smoke have been used to help clear airborne particles that can obscure the surgeon's vision. However, such systems require additional capital, including noisy and sophisticated motor-driven suctioning systems provided with expensive, high-efficiency filters. Furthermore, such systems lack the ability to efficiently and reliably suction both gas (smoke) and liquid fluids, because the liquids tend to clog the high-efficiency filters used to filter the smoke.

Therefore, a need exists for a mechanism to enable a surgeon to effectively and reliably remove both smoke and liquids from her field of view.

### SUMMARY

In accordance with an aspect of the disclosure, an electrosurgical system is provided and includes a gas control unit, a surgical instrument, an outflow conduit, and an inflow conduit. The surgical instrument has an elongated shaft and a nozzle. The nozzle is disposed at a distal end portion of the elongated shaft and defines a port. The outflow conduit has a proximal end portion coupled to the gas control unit and a distal end portion coupled to the elongated shaft. The distal end portion of the outflow conduit is in fluid communication with the port. The inflow conduit has a proximal end portion coupled to the gas control unit and a distal end portion configured for receipt in a small incision. The gas control unit is configured to induce suction in the inflow conduit and discharge an inert gas into the outflow conduit.

In aspects, gas control unit may be configured to adjust a suction pressure in the inflow conduit in proportion to a rate of flow of the inert gas to the outflow conduit.

In aspects, the surgical instrument may include an electrode coupled to the elongated shaft.

In aspects, the nozzle may define a central lumen having the electrode passing therethrough.

In aspects, the electrosurgical system may further include an electrosurgical generator in electrical communication with the electrode.

In aspects, the port may define a longitudinal axis that is offset from a central longitudinal axis defined by the nozzle.

In aspects, the longitudinal axis of the port may be parallel with the central longitudinal axis.

In aspects, the longitudinal axis of the port may be disposed at an angle of about 3 degree to about 7 degrees relative to the central longitudinal axis.

In aspects, the longitudinal axis of the port may be disposed at approximately a 5 degree angle relative to the central longitudinal axis.

In aspects, the port may be approximately 0.5 mm in diameter.

In aspects, the longitudinal axis of the port may extend radially outward relative to the central longitudinal axis.

In aspects, the one port may be a plurality of ports disposed circumferentially about a central longitudinal axis defined by the nozzle.

In aspects, the nozzle may define a central passageway and the ports may be disposed about the central passageway.

In aspects, the nozzle may taper in a distal direction.

In aspects, the nozzle may have an elliptical cone shape or a parabolic cone shape.

In accordance with an aspect of the disclosure, an electrosurgical instrument is provided and includes a handle, an elongated shaft extending distally from the handle, and a nozzle coupled to a distal end portion of the elongated shaft. The nozzle defines a plurality of discharge ports disposed circumferentially about a central longitudinal axis defined by the nozzle.

In aspects, each of the discharge ports may define a longitudinal axis that is offset from the central longitudinal axis.

In aspects, the longitudinal axis of each of the discharge ports may be parallel with the central longitudinal axis.

In aspects, the longitudinal axis of each of the discharge ports may be disposed at an angle of about 3 degrees to about 7 degrees relative to the central longitudinal axis.

In aspects, the longitudinal axis of each of the discharge ports may be disposed at approximately a 5 degree angle relative to the central longitudinal axis.

In aspects, each of the discharge ports may be approximately from about 0.3 mm to about 0.7 mm in diameter.

In aspects, the longitudinal axis of each of the discharge ports may extend radially outward relative to the central longitudinal axis.

In aspects, the nozzle may define a central passageway, and the discharge ports may be disposed about the central passageway.

In aspects, the electrosurgical instrument may further include a monopolar electrode movably received in the central passageway and configured to extend distally from the nozzle.

In aspects, the nozzle may taper in a distal direction.

In aspects, the nozzle may have an elliptical cone shape or a parabolic cone shape.

In aspects, the electrosurgical instrument may further include an outflow conduit extending proximally from the handle and in fluid communication with the ports.

In accordance with another aspect of the disclosure, a method of performing a surgical procedure is provided and includes positioning a nozzle of an electrosurgical instrument in a surgical site; treating tissue with the electrosurgical instrument; and discharging an inert gas from a port defined in the nozzle into the surgical site to disperse at least one of surgical smoke or blood.

In aspects, the method may further include positioning a distal end portion of an inflow conduit through a small incision in an abdominal wall; and removing the surgical smoke from within an abdominal cavity via the inflow conduit.

In aspects, removing the surgical smoke may include generating a suction pressure within the inflow conduit that is proportional to a rate of flow of the insert gas into the abdominal cavity from the port.

In aspects, treating the tissue may include transmitting electrosurgical energy from a monopolar electrode to the tissue. The monopolar electrode may extend distally from the nozzle.

In aspects, the port may include a plurality of ports extending radially outward relative to a central longitudinal axis defined by the nozzle.

As used herein, the term “distal” refers to the portion that is being described which is further from a surgeon, while the term “proximal” refers to the portion that is being described which is closer to a surgeon. Further, to the extent consistent, any of the aspects described herein may be used in conjunction with any or all of the other aspects described herein.

As used herein, the terms parallel and perpendicular are understood to include relative configurations that are substantially parallel and substantially perpendicular up to about +/− 10 degrees from true parallel and true perpendicular.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

### BRIEF DESCRIPTION OF THE DRAWINGS

Various aspects and features of the disclosure are described hereinbelow with reference to the drawings wherein like numerals designate identical or corresponding elements in each of the several views:

FIG. 1 is a front, schematic view illustrating an electrosurgical system including an electrosurgical generator, a gas control unit, and an electrosurgical instrument and a vent tube coupled to the gas control unit;

FIG. 2 is a side view illustrating a distal end portion of the electrosurgical instrument of FIG. 1;

FIG. 3 is a side, perspective view illustrating a nozzle of the electrosurgical instrument of FIG. 1;

FIG. 4 is a longitudinal cross-sectional view illustrating the nozzle of FIG. 3;

FIGS. 5A-5E are side, perspective views illustrating alternate embodiments of the nozzle of FIG. 3;

FIG. 6A is a side perspective view illustrating a distal end portion of an electrosurgical instrument including another embodiment of a monopolar electrode;

FIG. 6B is a side perspective view illustrating a distal end portion of an electrosurgical instrument including another embodiment of a monopolar electrode; and

FIG. 6C is a side perspective view illustrating a distal end portion of an electrosurgical instrument including another embodiment of a monopolar electrode.

### DETAILED DESCRIPTION

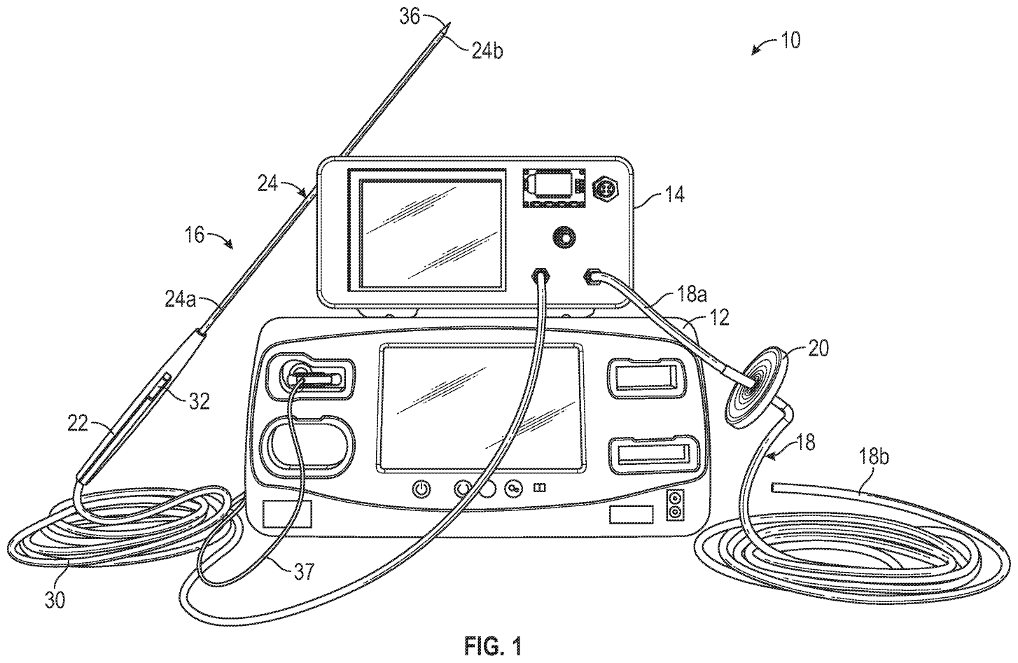

FIG. 1 illustrates an electrosurgical system 10 for performing a surgical procedure. The electrosurgical system 10 generally includes an electrosurgical generator 12, a gas control unit 14, and an electrosurgical instrument 16 operably coupled to both the generator 12 and the gas control unit 14. The gas control unit 14 is configured to both discharge an inert gas (e.g., carbon dioxide, nitrogen, and/or argon) and withdraw gas (e.g., surgical smoke, air, debris, etc.) via an inflow conduit 18, such as, for example, a vent tube.

The gas control unit 14 may include a processor (not explicitly shown) and a memory (not explicitly shown) having instructions stored thereon for execution by the processor. The instructions may include a predetermined flow rate of the inert gas. The instructions may include a plurality of flow rates with each corresponding to a particular type of surgical procedure. The preset flow rate may be from about 2 liters/minute (1/m) to about 7 l/m and, in some aspects, from about 2.5 l/m to about 5 l/m, and in other aspects, about 3.5 l/m. The flow rate of about 3.5 l/m was determined to be high enough to displace blood while being low enough to prevent bubbles from developing. If the flow rate is below 2 l/m, then the outflow pressure is too low to effectively displace blood, whereas if the flow rate is greater than about 7 l/m, the outflow pressure may create bubbles and blood may be splashed rather than displaced.

The inflow conduit 18 has a proximal end portion 18a coupled to the gas control unit 14 and a distal end portion 18b configured for receipt in a small incision. A filter 20 may be provided between the proximal and distal end portions 18a, 18b of the inflow conduit 18 to remove debris from surgical smoke generated at the surgical site. The gas control unit 14 is configured to induce suction in the inflow conduit 18 and adjust the suction pressure in the inflow conduit 18 in proportion to the rate of flow of the inert gas to the surgical instrument 16. In aspects, a flow rate and/or pressure sensor may be provided on the surgical instrument 16, or at any suitable location of the system 10, to assist in regulating the suction pressure provided by the inflow conduit 18 while maintaining the flow of inert gas to the surgical instrument 16 at a constant rate.

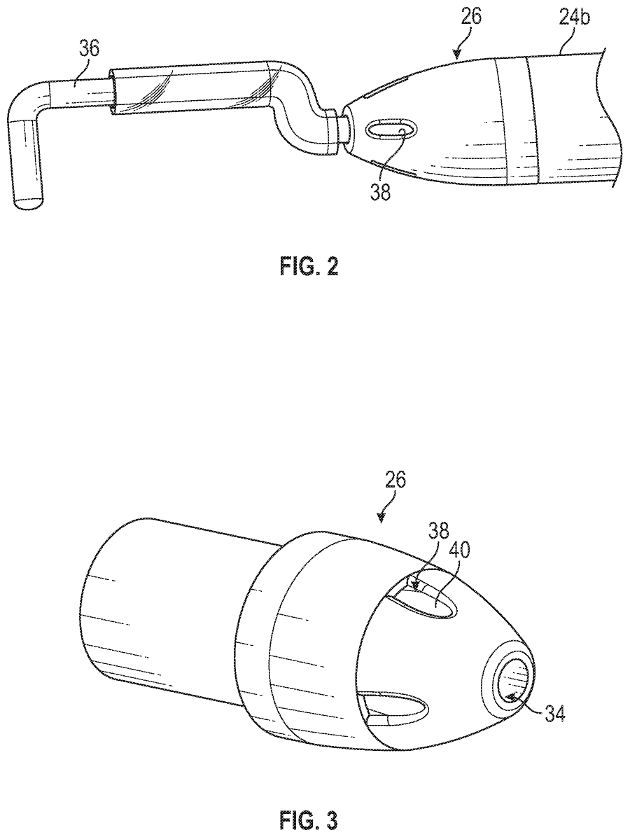

The surgical instrument 16 may be an electrosurgical instrument including a handle 22, an elongated shaft 24 extending distally from the handle 22, a nozzle 26 (FIG. 2) coupled to a distal end portion 24b of the elongated shaft 24, and an outflow conduit 30 (e.g., a tube) coupled between a proximal end portion 24a of the elongated shaft 24 and the gas control unit 14. The handle 22 may have a plurality of buttons or switches 32 for operating the surgical instrument 16. The handle 22 defines an elongated passageway (not explicitly shown) in fluid communication with an elongated passageway (not explicitly shown) defined through the elongated shaft 24 so that the inert gas may flow from the gas control unit 14 and distally through the surgical instrument 16.

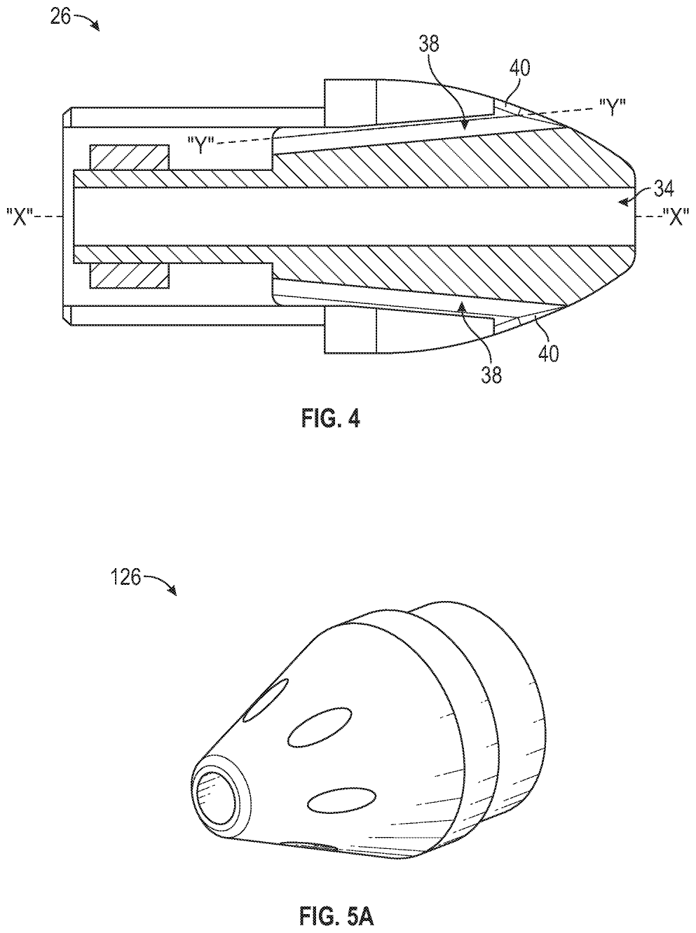

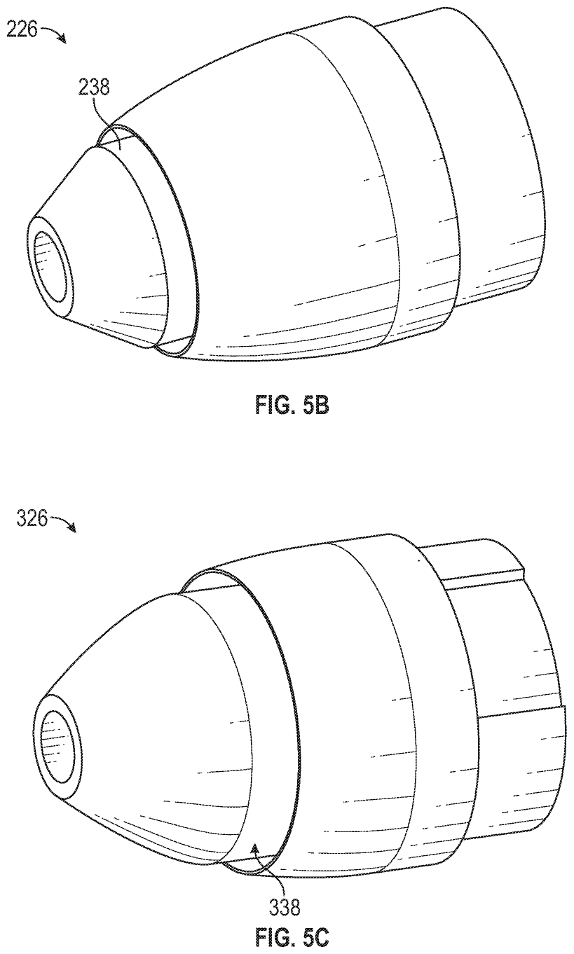

With reference to FIGS. 2-4, the nozzle 26 is received in the distal end portion 24b of the elongated shaft 24 and may be detachable therefrom or permanently affixed thereto. The nozzle 26 defines a central lumen 34 having an electrode 36, such as, for example, a monopolar electrode, received therein. The electrode 36 extends distally from the nozzle 26 and is in electrical communication, via one or more wires 37, with the electrosurgical generator 12 (FIG. 1). The nozzle 26 further defines a plurality of discharge ports 38 in fluid communication with the outflow conduit 30. As such, the discharge ports 38 of the nozzle 26 are configured to discharge the inert gas supplied by the gas control unit 14. The nozzle 26 may have any suitable number of ports, such as, from about 1 port to about 6 ports and, in some aspects, about 4 ports.

The ports 38 each have a distal opening 40 exposed to the ambient atmosphere. The distal opening 40 of each port 38 may be configured as an elongated cutout in an outer surface of the nozzle 26. Each of the ports 38 defines a longitudinal axis “Y” that is offset from a central longitudinal axis “X” defined by the nozzle 26. The central longitudinal axis “X” extends centrally through the central lumen 34, such that the ports 38 are circumferentially disposed about the central lumen 34. The longitudinal axis “Y” of each of the ports 38 extends radially outward relative to the central longitudinal axis “X” in a distal direction. The longitudinal axis “Y” of each of the ports 38 may be disposed at an angle of about 3 degree to about 7 degrees relative to the central longitudinal axis “X” and, in some aspects, the longitudinal axis “Y” of each of the ports 38 may be disposed at approximately a 5 degree angle relative to the central longitudinal axis “X” of the nozzle 26. In other aspects, the longitudinal axis “Y” of each of the ports 38 may be parallel with the central longitudinal axis “X.”

It was found that orientating the ports 38 between about 3 degrees and about 7 degrees relative to the central longitudinal axis “X” results in displacement of blood from a large area while concentrating the force. Orienting the ports 38 at an angle outside of the above range decreases the ports' 38 ability to displace blood and may also pose manufacturing obstacles.

The ports 38 may have a diameter from about 0.25 mm to about 1.0 mm and, in some aspects, approximately 0.5 mm. The nozzle 26 has a rounded outer profile and tapers in the distal direction. The nozzle 26 may have a generally elliptical cone shape, a parabolic cone shape, or any suitable shape.

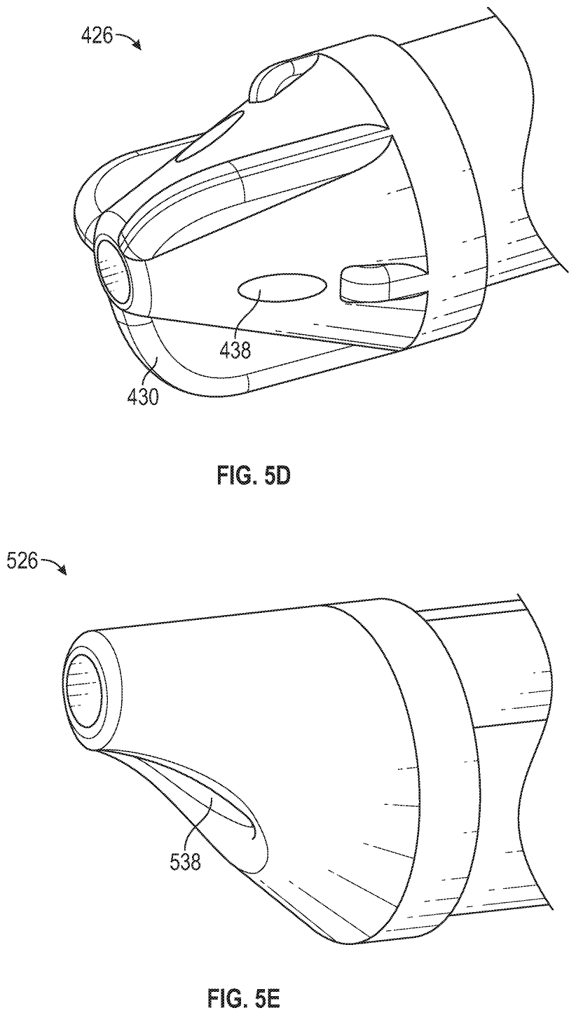

With brief reference to FIGS. 5A-5E, illustrated are various alternate shapes and structures for the nozzle 26. For example, FIG. 5A illustrates a nozzle 126 having a frusto-conical shape; FIG. 5B illustrates a nozzle 226 having one port 238 that extends continuously around a distal end of the nozzle 226; FIG. 5C illustrates a nozzle 326 having one port 338 that extends continuously around a middle region of the nozzle 326; FIG. 5D illustrates a nozzle 426 having a plurality of partitions 430 that separate each of a plurality of ports 438 from one another; and FIG. 5E illustrates a nozzle 526 having an oblique cone shape with an enlarged, single port 538.

In operation, the electrosurgical system 10 of the disclosure may be utilized to perform a minimally invasive surgical procedure or an open procedure. During, for example, a laparoscopic surgical procedure, the nozzle 26 of the electrosurgical instrument 16 and the distal end portion 18b of the inflow conduit 18 may be passed through a respective small incision in an abdominal wall and into an abdominal cavity. The monopolar electrode 36 of the electrosurgical instrument 16 is positioned in contact with a target piece of tissue and the electrosurgical generator 12 is activated to supply electrosurgical energy (e.g., RF energy) to the tissue via the monopolar electrode 36. During transmission of the electrosurgical energy, surgical smoke and/or blood may develop at the surgical site, which may inhibit the clinician's vision.

To improve the clinician's field of vision, the gas control unit 12 may be activated to simultaneously discharge inert gas (e.g., carbon dioxide) into the outflow tube 30 and generate a suction pressure in the inflow tube 18. The inert gas travels into the outflow tube 30 and passes through the elongated handle 22 and elongated shaft 24 of the electrosurgical instrument 16 and ultimately is discharged from the ports 38 in the nozzle 26. The flow of inert gas from the ports 38 may disperse blood and/or surgical smoke from the clinician's field of view. Due to the ports 38 being angled outwardly and disposed adjacent to and about the monopolar electrode 36, any surgical smoke and/or blood forming near the monopolar electrode 36 will be blown away by the inert gas. The inflow conduit 18 withdraws gas from within the abdominal cavity to remove surgical smoke therefrom and to maintain the pressure within the surgical cavity below a threshold pressure.

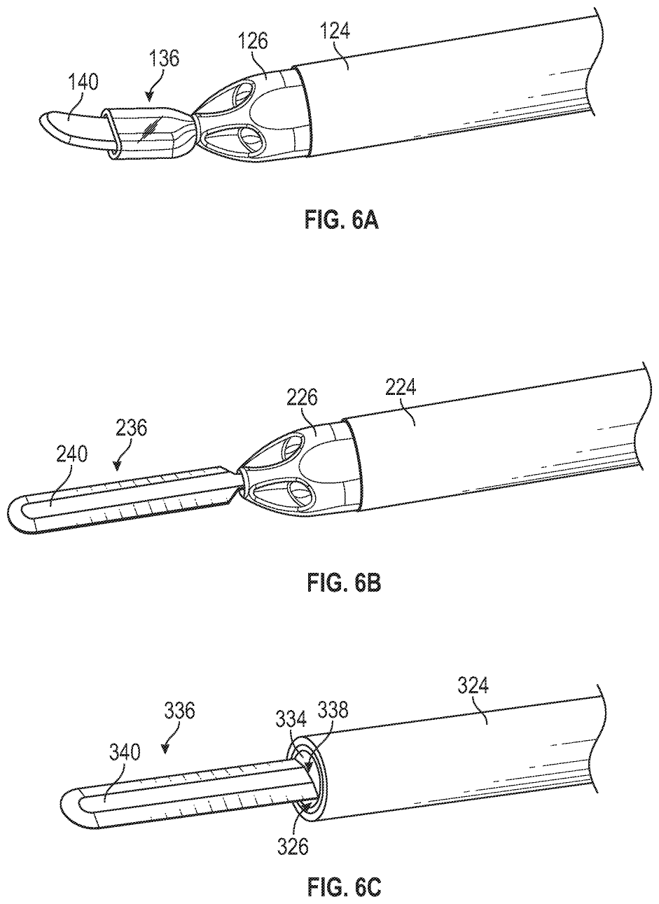

FIGS. 6A-6C illustrate alternative embodiments of the monopolar electrode 36. In particular, FIG. 6A illustrates a monopolar electrode 136 including a distal end portion 140 having a flat, elliptical shape and may curve along its length. The monopolar electrode 136 extends proximally through a nozzle 126, similar to the nozzle 26 (FIG. 2), for electrical connection with the generator 12 (FIG. 1). The nozzle 126 is coupled to an elongated shaft 124, similar to the elongated shaft 24 of FIG. 1.

FIG. 6B illustrates a monopolar electrode 236 more suitable for use in an open surgical procedure due to its reduced length when compared to the electrode 136 of FIG. 6A. The electrode 236 has a rigid, distal end portion 240 having a flat, elliptical shape and may be linear along its length. The electrode 236 extends proximally through a nozzle 226, similar to the nozzle 26 of FIG. 2, for electrical connection with the generator 12. The nozzle 226 is coupled to an elongated shaft 224 having a reduced length when compared to the elongated shaft 24 of FIG. 1.

FIG. 6C illustrates another monopolar electrode 336 more suitable for use in an open surgical procedure. The electrode 336 has a flexible distal end portion 340 having a flat, elliptical shape and may be linear along its length. The electrode 336 extends proximally through an alternative embodiment of a nozzle 326 for electrical connection with the generator 12. The nozzle 336 has a flat, distally-oriented face 334 defining a port 338 therethrough. The port 338 is configured to receive gas from the gas control unit 14 (FIG. 1) for expulsion therefrom during an open surgical procedure. The nozzle 326 is coupled to an elongated shaft 324 having a reduced length when compared to the elongated shaft 24 of FIG. 1.

The various embodiments disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the disclosure, and that such modifications and variations are also included within the scope of the disclosure. Accordingly, the subject matter of the disclosure is not limited by what has been particularly shown and described.

1. An electrosurgical system, comprising:
a gas control unit;
a surgical instrument having an elongated shaft, the elongated shaft having a nozzle disposed at a distal end portion thereof, the nozzle defining at least one port;
an outflow conduit having a proximal end portion coupled to the gas control unit and a distal end portion coupled to the elongated shaft and in fluid communication with the at least one port; and
an inflow conduit having a proximal end portion coupled to the gas control unit and a distal end portion configured for receipt in an incision, wherein the gas control unit is configured to induce suction in the inflow conduit and discharge a gas into the outflow conduit.

2. The electrosurgical system according to claim 1, wherein the gas control unit is configured to adjust a suction pressure in the inflow conduit in proportion to a rate of flow of the gas to the outflow conduit.

3. The electrosurgical system according to claim 2, wherein the rate of flow of the gas is from about 2 liters per minute to about 7 liters per minute.

4. The electrosurgical system according to claim 1, wherein the surgical instrument includes an electrode coupled to the elongated shaft.

5. The electrosurgical system according to claim 4, wherein the nozzle defines a central lumen having the electrode passing therethrough.

6. The electrosurgical system according to claim 1, wherein the at least one port defines a longitudinal axis that is offset from a central longitudinal axis defined by the nozzle.

7. The electrosurgical system according to claim 6, wherein the longitudinal axis of the at least one port is parallel with the central longitudinal axis.

8. The electrosurgical system according to claim 6, wherein the longitudinal axis of the at least one port is disposed at an angle of about 3 degree to about 7 degrees relative to the central longitudinal axis.

9. The electrosurgical system according to claim 8, wherein the longitudinal axis of the at least one port is disposed at approximately a 5 degree angle relative to the central longitudinal axis.

10. The electrosurgical system according to claim 9, wherein the at least one port is about 0.5 mm in diameter.

11. The electrosurgical system according to claim 6, wherein the longitudinal axis of the at least one port extends radially outward relative to the central longitudinal axis.

12. The electrosurgical system according to claim 1, wherein the at least one port is a plurality of ports disposed circumferentially about a central longitudinal axis defined by the nozzle.

13. The electrosurgical system according to claim 12, wherein the nozzle defines a central passageway, the plurality of ports disposed about the central passageway.

14. The electrosurgical system according to claim 1, wherein the nozzle tapers in a distal direction.

15. The electrosurgical system according to claim 14, wherein the nozzle has an elliptical cone shape or a parabolic cone shape.

16. An electrosurgical instrument, comprising:
a handle;
an elongated shaft extending distally from the handle;
a nozzle coupled to a distal end portion of the elongated shaft and defining a plurality of discharge ports disposed circumferentially about a central longitudinal axis defined by the nozzle.

17. The electrosurgical instrument according to claim 16, wherein each of the plurality of discharge ports defines a longitudinal axis that is offset from the central longitudinal axis.

18. The electrosurgical instrument according to claim 17, wherein the longitudinal axis of each of the plurality of discharge ports is parallel with the central longitudinal axis.

19. The electrosurgical instrument according to claim 17, wherein the longitudinal axis of each of the plurality of discharge ports is disposed at an angle of about 3 degree to about 7 degrees relative to the central longitudinal axis.

20. The electrosurgical instrument according to claim 19, wherein the longitudinal axis of each of the plurality of discharge ports is disposed at about a 5 degree angle relative to the central longitudinal axis.

21. The electrosurgical instrument according to claim 20, wherein each of the plurality of discharge ports is from about 0.3 mm to about 0.7 mm in diameter.

22. The electrosurgical instrument according to claim 17, wherein the longitudinal axis of each of the plurality of discharge ports extends radially outward relative to the central longitudinal axis.

23. The electrosurgical instrument according to claim 16, wherein the nozzle defines a central passageway, the plurality of discharge ports being disposed about the central passageway.

24. The electrosurgical instrument according to claim 23, further comprising a monopolar electrode movably received in the central passageway and configured to extend distally from the nozzle.

25. The electrosurgical instrument according to claim 16, wherein the nozzle tapers in a distal direction.

26. The electrosurgical instrument according to claim 25, wherein the nozzle has an elliptical cone shape or a parabolic cone shape.

27. The electrosurgical instrument according to claim 16, further comprising an outflow conduit extending proximally from the elongated handle and in fluid communication with the plurality of ports.
